(19) United States
(12) Patent Application Publication
(10) Pub. No.: US 2018/0185430 A1
(43) Pub. Date: Jul. 5, 2018

(54) Herbal Composition for Shrimp Disease

(71) Applicants: Yujia Zhu, Tempe, AZ (US); Yahuan Cui, Tempe, AZ (US)

(72) Inventors: Yujia Zhu, Tempe, AZ (US); Yahuan Cui, Tempe, AZ (US)

(21) Appl. No.: 15/396,165

(22) Filed: Dec. 30, 2016

(51) Int. Cl. A61K 36/718; A61K 47/46; A61K 36/28; A61K 36/34; A61K 47/26

(52) U.S. Cl. CPC A61K 36/718; A61K 47/46; A61K 47/26; A61K 36/34; A61K 36/28

(57) ABSTRACT

A composition of herbal medicine which can be used for shrimp disease treatment, includes the following ingredients: Lobelia chinensis Lour, Folium Loropetali, composition X and fishmeal, crude protein, soymeal, powder, dextrin, glucose and minerals. The composition is used in manufacturing three different medicaments and for the different medicaments, the composition X includes one or more ingredients of astragalus, vitexin flower, elephantopus flower and Coptis chinensis leaf. The composition and the medicaments are effective in treating the shrimp infectious disease, protozoa and improving the shrimp immune system.

### TECHNICAL FIELD

The present invention relates to the traditional Chinese medicine composition, which is prevention and treatment of shrimp disease. The present invention also includes preparation method and application of the composition.

### BACKGROUND OF THE INVENTION

According to the estimate from Food and Agriculture Organization of the United Nations (FAO), because the offshore fishing has decreased by overexploitation, half of the global seafood consumption will be from aquaculture by 2020. While the market demand is promising for the shrimp farming industry, shrimp disease is a serious threat. One example is that the outbreak of the Acute Hepatopancreatic Necrosis Disease (AHPND) in Thailand in between 2012 and 2014 has cost a $5.01 billion lost and the production was dropped by 60%. The intensive shrimp farming and the export-and-import of shrimp and larvae even boost the outbreak frequency and spreading speed. In China, the incidence of penaeus vannamei red body disease in Guangdong Province was 95% and caused 100% mortality rate. Most of the shrimp diseases have over 80% mortality rate which are very high. However, for many of such diseases, there is no effective treatment at present.

There are more than 500 kinds of shrimp medicine in the market, including disinfectant, environment modifiers, insecticide, insect repellant. But none of these can insure a high survival rate. Even some farmers break rules and use antibiotics but it is still much less effective than expected. The overuse of antibiotics causes eco-environmental and food safety problems. It also causes resistance to drugs and vicious cycle of shrimp disease. Research from Ministry of Agriculture in China shows that Chinese herbal medicine has great effects and prospect in aquaculture and encourage researches on it. For the safe and sustainable development of aquaculture, it is fruitful to research and develop herbal medicine for shrimp disease, which has no toxic and side effects, no residue and does not cause drug resistance.

The present invention is a composition that can prevent and treat shrimp disease is composed of Lobelia chinensis Lour, elephantopus and Coptis chinensis and adjuvant. Adjuvant includes fishmeal, crude protein, soymeal, flour, dextrin, glucose and mineral. Lobelia chinensis Lour has the capability of deswelling, clearing heat and detoxification. Folium Loropetali has the capability of clearing the heat, detoxification and hemostasis. Research from modern chemistry and pharmacology shows that Lobelia chinensis Lour has strong antiseptic and antibacterial effects; elephantopus has strong effects of antibiosis, anti-cytotoxicity and anti-tumor. The Coptis chinensis is used in concert with Lobelia chinensis Lour for anti-bacteria and anti-protozoa. The composition has great effects of both clearing heat and detoxification from traditional Chinese medicine, and antibacterial and immune regulation from modern medicine, which is very effective treatment of shrimp infectious disease.

### BRIEF SUMMARY OF THE INVENTION

An objective of the invention is to provide a composition of preventing and curing shrimp disease based on prescription. The composition has effects of resisting shrimp infectious disease and improving immunity.

Another objective of the invention is to provide preparation method of the composition mentioned above.

Another objective of the invention is to provide application of the composition mentioned above.

All the objectives of the invention mentioned above can be achieved by the following techniques:

The present convention is a composition used to prevent and cure shrimp disease. The composition includes Lobelia chinensis Lour., X and adjuvants. The ingredients in weights include 400-500 portion of Lobelia chinensis Lour., 200-400 portion of X and 100-700 of the adjuvants. The X includes elephantopus flower, Coptis chinensis leaf. The adjuvant is a mixture of fishmeal, crude protein, soymeal, glucose, dextrin and minerals.

All the ingredients in the prescription can be used for antibacterial. Table 1 is given to show the ingredients and their effective bacteria.

The Lobelia chinensis Lour. is used for clearing heat, detoxification, deswelling. Immunoregulation is another effects of the Lobelia chinensis Lour. The elephantopus flower is used for clearing heat, detoxification, deswelling and diuresis. Coptis chinensis leaf is used for clearing heat and dampness, detoxification and more importantly suppress the harmful protozoa including amoebas, trypanosomatid, etc.

### BRIEF DESCRIPTION OF DRAWINGS

FIG. 1 shows the comparison on MIC tests in example 4.

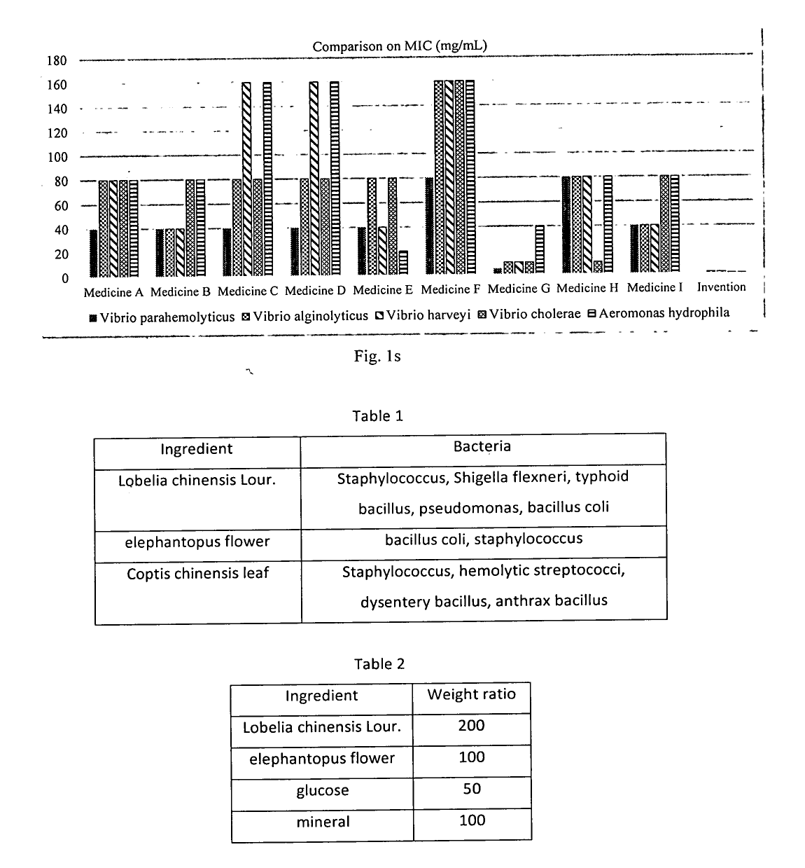

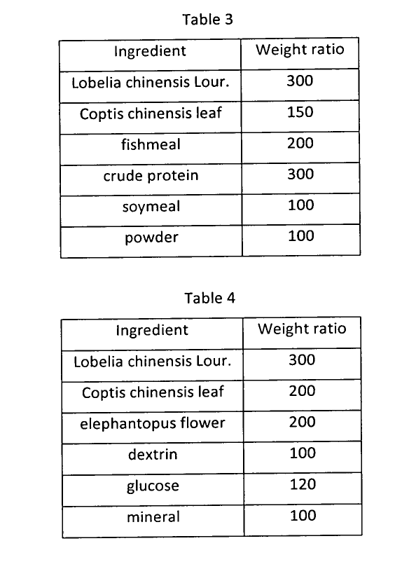

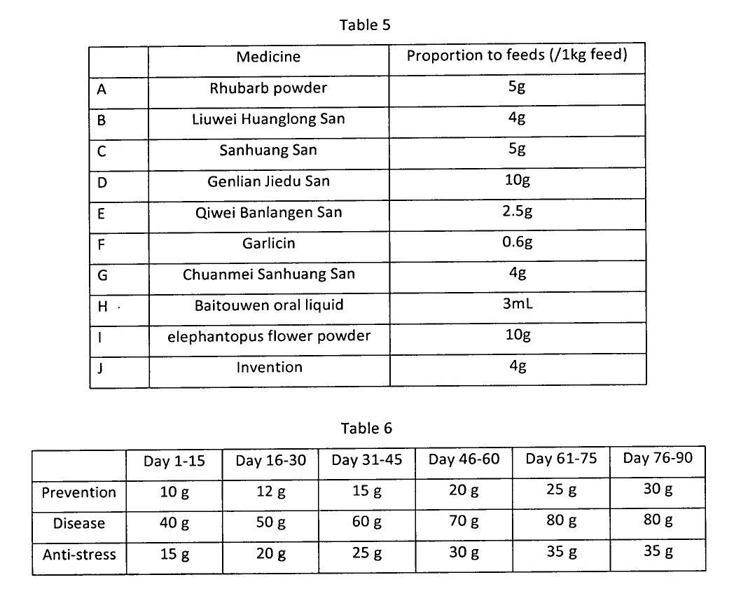

Table 1 shows the ingredients of the invention and the corresponding effective bacteria.

Table 2 shows the ingredients of the composition in Example 1.

Table 3 shows the Ingredients of the composition in Example 2.

Table 4 shows the ingredients of the composition in Example 3.

Table 5 shows the medicaments used in the MIC test in Example 4.

Table 6 shows the dosage of composition A per treatment for 10 thousand larvae shrimp)

### DETAILED DESCRIPTION OF THE INVENTION

The preparation of the composition includes following steps:
(1) Apply aqueous extract to Lobelia chinensis Lour separately. Use 8-18 times amount of water to extract twice and 1-2 hours every time. Filter and combine the filtrate. Decompress and condense the mixed filtrate under 50-80° C. to paste. Add ethanol and quickly stir the paste to 60%-80% alcohol content. Leave the paste for a night. Filter the supernatant liquid and retrieve the ethanol. Decompress and condense the mixed filtrate under 50-80° C. to paste. The paste obtained is the paste A.
(2) Extract from X twice with 8-10 times amount of ethanol one with 90% ethanol and one with 70% ethanol. Each extraction takes 1-2 hours. Filer the extracts. Mix the extracts (i.e. extracts with different ethanol). Decompress and condense the mixed filtrate under 50-80° C. to paste and retrieve the ethanol. The obtained paste is paste B.
(3) Combined the paste A and paste B to obtain paste C.
(4) Heat water to 80-90° C. Add glucose, minerals and quickly stir to completely dissolve. Add paste C obtained from step (3). Stir and keep the temperature at 50° C. Add antiseptic and stir. Obtain the composition A.
(5) Mix fishmeal, crude protein soymeal and powder. Add paste C obtained from step (3). Stir and add antiseptic, obtain composition B.
(6) Heat water to 80-90° C. Add dextrin, glucose and minerals. Quickly stir to completely dissolve Add paste C obtained (3). Stir and keep in 50° C. Add antiseptic and stir. Obtain the composition C.

Preparation with the compositions to medicaments takes following steps:

1. Cool down the composition A and fill in bottle and seal. Obtain a liquid medicament for shrimp disease treatment.
2. Granulate the composition B. Oven dry in 70° C. Separately pack and seal. Obtain the particle medicament for shrimp disease treatment.
3. Cool down the composition C and fill in bottle and seal. Obtain a medicament in paste for shrimp disease treatment.

### DETAILED EXAMPLES (THE EXAMPLES DO NOT GIVE ANY LIMITATION TO THE PRESENT INVENTION)

### Example 1

A Chinese herbal medicine based composition of which main ingredients includes Lobelia chinensis Lour., elephantopus flower, glucose, minerals. The weight ratio of the ingredients are shown in Error! Reference source not found.

The preparation of the composition follows steps below.
(1) Prepare extraction: Apply aqueous extract to Lobelia chinensis Lour. Use 8-18 times amount of water to extract twice and 1-2 hours every time. Filter and combine the filtrate. Decompress and condense the mixed filtrate under 50-80° C. to paste. Add ethanol and quickly stir the paste to 60%-80% alcohol content. Leave the paste for a night. Filter the supernatant liquid and retrieve the ethanol. Decompress and condense the mixed filtrate under 50-80° C. to paste. Extract from elephantopus flower, twice with 8-10 times amount of ethanol one with 90% ethanol and one with 70% ethanol. Each extraction takes 1-2 hours. Filter the extracts. Mix the extracts (i.e. extracts with different ethanol). Decompress and condense the mixed filtrate under 50-80° C. to paste and retrieve the ethanol. Mix the two pastes.
(2) Dissolve: Heat water to 80-90″C. Add glucose, minerals and quickly stir to completely dissolve. Add paste obtained from step (1). Stir and add antiseptic. Keep in 50° C. Add antiseptic and stir. Obtain the composition.

Cool down the composition. Fill into the bottle and seal with filling machine:

### Example 2

A Chinese herbal medicine based composition of which main ingredient includes Lobelia chinensis Lour., Coptis chinensis leaf, fishmeal, crude protein, soymeal, powder. The weight ratio of the ingredients are shown in Error! Reference source not found.

The preparation of the composition follows steps below.
(1) Prepare extraction: Apply aqueous extract to Lobelia chinensis Lour. Use 8-18 times amount of water to extract twice and 1-2 hours every time. Filter and combine the filtrate. Decompress and condense the mixed filtrate under 50-80° C. to paste. Add ethanol and quickly stir the paste to 60%-80% alcohol content. Leave the paste for a night. Filter the supernatant liquid and retrieve the ethanol. Decompress and condense the mixed filtrate under 50-80° C. to paste. Extract from Coptis chinensis leaf twice with 8-10 times amount of ethanol one with 90% ethanol and one with 70% ethanol. Each extraction takes 1-2 hours. Filer the extracts. Mix the extracts (i.e. extracts with different ethanol). Decompress and condense the mixed filtrate under 50-80° C. to paste and retrieve the ethanol. Mix the two pastes.
(2) Mix fishmeal, crude protein, soymeal and powder. Add the paste from step (1), stir. Add antiseptic, stir again.

Preparation: Granulate into particles. Oven dry in 70° C. Pack and seal.

### Example 3

A Chinese herbal medicine based composition of which main ingredient includes Lobelia chinensis Lour., Coptis chinensis leaf, elephantopus flower, dextrin, glucose, mineral. The weight ratio of the ingredients are shown in Error! Reference source not found.

The preparation of the composition follows steps below.
(1) Prepare extraction: Apply aqueous extract to Lobelia chinensis Lour. Use 8-18 times amount of water to extract twice and 1-2 hours every time. Filter and combine the filtrate. Decompress and condense the mixed filtrate under 50-80° C. to paste. Add ethanol and quickly stir the paste to 60%-80% alcohol content. Leave the paste for a night. Filter the supernatant liquid and retrieve the ethanol. Decompress and condense the mixed filtrate under 50-80° C. to paste. Extract from Coptis chinensis leaf and elephantopus flower twice with 8-10 times amount of ethanol one with 90% ethanol and one with 70% ethanol. Each extraction takes 1-2 hours. Filer the extracts. Mix the extracts (i.e. extracts with different ethanol). Decompress and condense the mixed filtrate under 50-80° C. to paste and retrieve the ethanol. Mix the two pastes.
(2) Dissolve: Heat water to 80-90° C. Add dextrin, glucose and minerals. Quickly stir to completely dissolve Add paste from step (1). Stir and keep in 50° C. Add antiseptic and stir. Obtain the composition.
(3) Preparation: Fill the liquid compound and seal.

### Example 4 Minimum Inhibition Test

The test compares the invention (medicament of composition A) to 10 commonly used Chinese herbal medicine based medicaments. The 10 medicines are listed in Error! Reference source not found. The medicines are mixed with feeds and applied to the shrimps infected by Vibrio parahemolyticus, Vibrio alginolyticus, Vibrio harveyi, Vibrio vulnificus and Aeromonas hydrophila respectively. The proportion of between the medicine and the feed are shown in the second column of Error! Reference source not found.

Every medicament was made into 0.8 g/ml medicine liquor with respective solvent. Sterilize with ultra-high pressure. Apply serial two-fold dilution 11 times and obtain solvents with 11 different concentrations.

Use streak culture on agar plates to recover the bacteria for 24 hours. Inoculate the bacteria to the tube with 2216E broth culture medium. Place in the constant temperature incubator at 37° C. to logarithmic phase.

Use the 2216E broth as a bench mark solution, measure the OD value of the bacterial suspension with 600 nm wavelength, calculate the density of the bacteria, dilute the bacterial suspension to 1×106 CFU/ml.

Draw 100 μl medicine liquor and bacteria suspension respectively under aseptic process to the No. 1-9 wells of the 96 well plate. Add 100 μl invention solution and 100 μl bacterial suspension to the No. 12 well as a negative control. Place the in the constant temperature incubator under 37° C. for 20 hours.

The minimum inhibition concentration (MIC) of the invention for the Vibrio parahemolyticus, Aeromonas hydrophila, Vibrio vulnificus is 0.313 g/L; for Vibrio alginolyticus and Vibrio harveyi the MIC is 1.250 g/L. The MIC of other conventional medicines are at least 5 g/L and most of them are over 40 g/L. More detailed results are shown in Error! Reference source not found.

### Example 5 Clinical Cases

Case 1 Client: Shrimp farm owner Shao from Shuidong Town, Maoming City, Guangdong Province

Shao had a 3.5-acre higher-place pond in winter canopy and 180 thousand larvae shrimp per acre. From February to May 2016, Shao used the composition A, B and C for prevention, one treatment is for two consecutive days, one time per day, four days between each treatment. With the first harvest of 1250 kilograms on May 6, 2016 and the second harvest of 3750 kilograms on Jun. 2, 2016, the total output was 5000 kilograms, over 1000 kilograms per acre. The survival rate was 85%!

Case 2 Client: Shrimp farm owner Zhou from Lianxi Town, Zhuhai City, Guangdong Province

Zhou had three 5-acre pond in winter canopy and 100 thousand larvae shrimp per acre. Zhou used the present composition for prevention, one treatment is for two consecutive days, one time per day, four days between each treatment. The total output in April 2016 was 7500 kilograms. It took only 120 days from larvae shrimp to harvest and the average cost per kilogram for preventing was only one CNY.

### Clinical Usage and Dosage

### 1 Composition A:

In the shrimp feeding process, feed shrimp with composition A before the first feeding in the morning, then feed shrimp with regular feed after 15 minutes.

The dosage of composition A for 10 thousand larvae shrimp is given in Table 6.

*For prevention and anti-stress, one treatment per day. For infectious disease, two treatments per day.

### 2 Composition B:

2.1 For prevention: Use composition B once every five days. Using 400 ml composition B with one kilogram composition A, feed shrimp before the first feed in the morning once a day for two consecutive days.

2.2 For Anti-stress: Using 400 ml composition B with one kilogram composition A, feed shrimp before the first feed in the morning once a day till it turns better.

2.3 For infectious disease: Stop normal feeding. Using 400 ml composition B with one kilogram composition A, feed shrimp once in the morning and once in the evening till it turns better.

1. A composition used as prophylaxis and/or therapy of shrimp disease, the composition containing: Lobelia chinensis Lour, elephantopus and Coptis chinensis and adjuvant.

2. A composition as claimed in claim 1, wherein the adjuvant containing glucose and minerals

3. A composition as claimed in claim 1, wherein the composition is in the form of a granular formulation, liquid formulation and paste formulation.

4. A composition as claimed in claim 1, wherein the composition is used in preparing and/or manufacturing shrimp disease prophylactic and/or therapeutic medicaments.

5. A process for preparing the composition useful as prophylactic and/or therapeutic of shrimp disease, said process comprising: i) obtaining extracts from Lobelia chinensis Lour, obtaining filtrate by filtering the extracts, obtaining paste from the filtrate, stirring the paste and adding in ethanol, filtering the supernatant liquid and retrieving the ethanol, condensing the supernatant liquid to a first paste; obtaining extracts from elephantopus and Coptis chinensis with ethanol; filtering the extract with ethanol; condensing the filtrate to a second paste; combining said first paste and said second paste to a third paste; ii) heating water to 80-90° C., adding in glucose, minerals and quickly stir to completely dissolve, adding in paste C obtained from step i), stirring and keeping the temperature at 50° C., adding in antiseptic and stirring, obtaining the composition A; iii) mixing fishmeal, crude protein soymeal and powder, adding in the third paste obtained from step i), stirring and adding in antiseptic, obtaining composition B; iv) heating water to 80-90° C., adding in dextrin, glucose, mineral, stirring to completely dissolve, adding in the third paste obtained from step i), stirring and keeping the temperature at 50° C., adding in antiseptic and stirring, obtaining the composition C.
